(19) United States
(12) Patent Application Publication
(10) Pub. No.: US 2024/0036037 A1
(43) Pub. Date: Feb. 1, 2024

(54) FLUIDIC CARTRIDGE MODULE, BIOSENSOR DEVICE, METHOD OF DETECTING ANALYTE IN SAMPLE

(71) Applicant: Taiwan Semiconductor Manufacturing Company, Ltd., Hsinchu (TW)

(72) Inventors: Yi-Hsing Hsiao, Hsinchu City (TW); Yu-Jie Huang, Kaohsiung City (TW); Tung-Tsun Chen, Hsinchu City (TW)

(73) Assignee: Taiwan Semiconductor Manufacturing Company, Ltd., Hsinchu (TW)

(21) Appl. No.: 17/873,146

(22) Filed: Jul. 26, 2022

(51) Int. Cl. G01N 33/543; G01N 33/569; G01N 27/414; B01L 3/00

(52) U.S. Cl. CPC G01N 33/54373; G01N 33/56983; G01N 27/4145; B01L 3/502; B01L 2300/0636; B01L 2200/16; B01L 2200/04; B01L 2300/021

(57) ABSTRACT

A fluidic cartridge module includes a casing, a biosensor package, and a fluidic channel. The casing includes a sample inlet and a buffer inlet, a biosensor package disposed in the casing and comprising a sensor array and a reference electrode. The fluidic channel is disposed over the biosensor package and connected to the sample inlet and the buffer inlet, wherein the fluidic channel includes a first opening aligned with the sensor array and a second opening aligned with the reference electrode.

### BACKGROUND

Biosensors are devices for sensing and detecting biomolecules and operate on the basis of electronic, electrochemical, optical, and mechanical detection principles. Biosensors that include transistors are sensors that electrically sense charges, photons, and mechanical properties of bio-entities or biomolecules. The detection can be performed by detecting the bio-entities or biomolecules themselves, or through interaction and reaction between specified reactants and bio-entities/biomolecules. Such biosensors can be manufactured using semiconductor processes, can quickly convert electric signals, and can be easily applied to integrated circuits (ICs) and MEMS.

The interaction of the biological sample itself and the biosensor can be a challenge. Typically, a fluid containing the biological sample is pipetted directly over the sensing portion of the biosensor. This method leads to a large portion of the fluid sample not being used, and is time consuming to manually load each sensing area. Other fluid delivery systems involve the use of pumps that deliver fluid through tubing to the sensor area. Such systems are highly reliant on the precise operation of the pumps and any valves being used, and are difficult to maintain as they become smaller.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 illustrates a schematic view of a biosensor device according to some exemplary embodiments of the present disclosure.

FIG. 2 illustrates an exploded view of a biosensor device according to some exemplary embodiments of the present disclosure.

FIG. 3 illustrates a schematic view of a fluidic cartridge module according to some exemplary embodiments of the present disclosure.

FIG. 4 illustrates an exploded view of the fluidic cartridge module in FIG. 3.

FIG. 5 illustrates a schematic floor plan diagram of a biosensor chip according to some exemplary embodiments of the present disclosure.

FIG. 6 illustrates a schematic cross sectional view of a sensor of a biosensor chip according to some exemplary embodiments of the present disclosure.

FIG. 7 illustrates a schematic top view of a first channel frame of a fluidic cartridge module according to some exemplary embodiments of the present disclosure.

FIG. 8 illustrates a schematic bottom view of the first channel frame in FIG. 7.

FIG. 9 illustrates a schematic top view of a second channel frame of a fluidic cartridge module according to some exemplary embodiments of the present disclosure

FIG. 10 illustrates a schematic view of a fluidic cartridge module coupled to a processor according to some exemplary embodiments of the present disclosure.

FIG. 11 to FIG. 13 illustrate schematic operating scenarios of a fluidic cartridge module according to some exemplary embodiments of the present disclosure.

FIG. 14 illustrates a schematic view of a part of a biosensor device according to some exemplary embodiments of the present disclosure.

FIG. 15 illustrates an exploded view of the fluidic cartridge module according to some exemplary embodiments of the present disclosure.

FIG. 16 illustrates a flow diagram of an exemplary method of detecting an analyte in a sample.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of embodiments in accordance with the disclosure; the methods, devices, and materials are now described.

The acronym “FET,” as used herein, refers to a field effect transistor. A very common type of FET is referred to as a metal oxide semiconductor field effect transistor (MOSFET). Historically, MOSFETs have been planar structures built in and on the planar surface of a substrate such as a semiconductor wafer. But recent advances in semiconductor manufacturing have resulted in three-dimensional, of fin-based, MOSFET structures.

The term “bioFET” refers to a FET that includes a layer of immobilized capture reagents that act as surface receptors to detect the presence of a target analyte of biological origin. A bioFET is a field-effect sensor with a semiconductor transducer, according to an embodiment. One advantage of bioFETs is the prospect of label-free operation. Specifically, bioFETs enable the avoidance of costly and time-consuming labeling operations such as the labeling of an analyte with, for instance, fluorescent or radioactive probes. One specific type of bioFET described herein is a dual-gate back-side sensing bioFET, for example. The analytes for detection by a BioFET will normally be of biological origin, such as—without limitation—proteins, carbohydrates, lipids, tissue fragments or portions thereof. However, in a more general sense a BioFET is part of a broader genus of FET sensors that may also detect any chemical compound (known in the art as a ChemFET), or any other element, including ions, such as protons or metallic ions (known in the art as an ISFET). This disclosure is meant to apply to all types of FET-based sensors (“FET Sensor”). One specific type of FET Sensor herein is a Dual-Gate Back Side Sensing FET Sensor (“DG BSS FET Sensor”).

In addition, “S/D” refers to the source/drain junctions that form two of the four terminals of a FET. The expression “high-k” refers to a high dielectric constant. In the field of semiconductor device structures and manufacturing processes, high-k refers to a dielectric constant that is greater than the dielectric constant of SiO2 (i.e., greater than 3.9). The term “analysis” generally refers to a process or step involving physical, chemical, biochemical, or biological analysis that includes, but is not limited to, characterization, testing, measurement, optimization, separation, synthesis, addition, filtration, dissolution, or mixing.

The term “assay” generally refers to a process or step involving the analysis of a chemical or a target analyte and includes, but is not limited to, cell-based assays, biochemical assays, high-throughput assays and screening, diagnostic assays, pH determination, nucleic acid hybridization assays, polymerase activity assays, nucleic acid and protein sequencing, immunoassays (e.g., antibody-antigen binding assays, ELISAs, and iqPCR), bisulfite methylation assays for detecting methylation pattern of genes, protein assays, protein binding assays (e.g., protein-protein, protein-nucleic acid, and protein-ligand binding assays), enzymatic assays, coupled enzymatic assays, kinetic measurements (e.g., kinetics of protein folding and enzymatic reaction kinetics), enzyme inhibitor and activator screening, chemiluminescence and electrochemiluminescence assays, fluorescent assays, fluorescence polarization and anisotropy assays, absorbance and colorimetric assays (e.g., Bradford assay, Lowry assay, Hartree-Lowry assay, Biuret assay, and BCA assay), chemical assays (e.g., for the detection of environmental pollutants and contaminants, nanoparticles, or polymers), and drug discovery assays. The module, device, apparatus, and methods described herein may use or adopt one or more of these assays to be used with any of the FET Sensor described designs.

The term “liquid biopsy” generally refers to a biopsy sample obtained from a subject's bodily fluid as compared to a subject's tissue sample. The ability to perform assays using a body fluid sample is oftentimes more desirable than using a tissue sample. The less invasive approach using a body fluid sample has wide ranging implications in terms of patient welfare, the ability to conduct longitudinal disease monitoring, and the ability to obtain expression profiles even when tissue cells are not easily accessible, e.g., in the prostate gland. Assays used to detect target analytes in liquid biopsy samples include, but are not limited to, those described above.

The term “identification” generally refers to the process of determining the identity of a target analyte based on its binding to a capture reagent whose identity is known. The term “measurement” generally refers to the process of determining the amount, quantity, quality, or property of a target analyte based on its binding to a capture reagent. The term “quantitation” generally refers to the process of determining the quantity or concentration of a target analyte based on its binding to a capture reagent. The term “detection” generally refers to the process of determining the presence or absence of a target analyte based on its binding to a capture reagent. Detection includes but is not limited to identification, measurement, and quantitation. The term “chemical” refers to a substance, compound, mixture, solution, emulsion, dispersion, molecule, ion, dimer, macromolecule such as a polymer or protein, biomolecule, precipitate, crystal, chemical moiety or group, particle, nanoparticle, reagent, reaction product, solvent, or fluid any one of which may exist in the solid, liquid, or gaseous state, and which is typically the subject of an analysis.

The term “reaction” refers to a physical, chemical, biochemical, or biological transformation that involves at least one chemical and that generally involves (in the case of chemical, biochemical, and biological transformations) the breaking or formation of one or more bonds such as covalent, noncovalent, van der Waals, hydrogen, or ionic bonds. The term includes typical chemical reactions such as synthesis reactions, neutralization reactions, decomposition reactions; displacement reactions, reduction-oxidation reactions, precipitation, crystallization, combustion reactions, and polymerization reactions, as well as covalent and noncovalent binding, phase change, color change, phase formation, crystallization, dissolution, light emission, changes of light absorption or emissive properties, temperature change or heat absorption or emission, conformational change, and folding or unfolding of a macromolecule such as a protein.

The term “capture reagent” as used herein, is a molecule or compound capable of binding the target analyte or target reagent, which can be directly or indirectly attached to a substantially solid material. The capture agent can be a chemical, and specifically any substance for which there exists a naturally occurring target analyte (e.g., an antibody, polypeptide, DNA, RNA, cell, virus, etc.) or for which a target analyte can be prepared, and the capture reagent can bind to one or more target analytes in an assay. “Target analyte” as used herein, is the substance to be detected in the test sample using the present invention. The target analyte can be a chemical, and specifically any substance for which there exists a naturally occurring capture reagent (e.g., an antibody, polypeptide, DNA, RNA, cell, virus, etc.) or for which a capture reagent can be prepared, and the target analyte can bind to one or more capture reagents in an assay. “Target analyte” also includes any antigenic substances, antibodies, and combinations thereof. The target analyte can include a protein, a peptide, an amino acid, a carbohydrate, a hormone, a steroid, a vitamin, a drug including those administered for therapeutic purposes as well as those administered for illicit purposes, a bacterium, a virus, and metabolites of or antibodies to any of the above substances.

The term “Test sample” as used herein, means the composition, solution, substance, gas, or liquid containing the target analyte to be detected and assayed using the present invention. The test sample can contain other components besides the target analyte, can have the physical attributes of a liquid, or a gas, and can be of any size or volume, including for example, a moving stream of liquid or gas. The test sample can contain any substances other than the target analyte as long as the other substances do not interfere with the binding of the target analyte with the capture reagent or the specific binding of the first binding member to the second binding member. Examples of test samples include, but are not limited to naturally-occurring and non-naturally occurring samples or combinations thereof. Naturally-occurring test samples can be synthetic or synthesized. Naturally-occurring test samples include body or bodily fluids isolated from anywhere in or on the body of a subject, including but not limited to, blood, plasma, serum, urine, saliva or sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid and combinations thereof, and environmental samples such as ground water or waste water, soil extracts, air, and pesticide residues or food-related samples.

Detected substances can include, e.g., nucleic acids (including DNA and RNA), hormones, different pathogens (including a biological agent that causes disease or illness to its host, such as a virus (e.g., covid-19, SARS, H1N1), a protozoan (e.g., Plasmodium-causing malaria), or a bacteria (e.g., E. coli or Mycobacterium tuberculosis)), proteins, antibodies, various drugs or therapeutics or other chemical or biologic or substances, including hydrogen or other ions, non-ionic molecules or compounds, polysaccharides, small chemical compounds such as chemical combinatorial library members, and the like. Detected or determined parameters may include but are not limited to, e.g., pH changes, lactose changes, changing concentration, particles per unit time where a fluid flows over the device for a period of time to detect particles, e.g., particles that are sparse, and other parameters.

As used herein, the term “immobilized,” when used with respect to, e.g., a capture reagent, includes substantially attaching the capture reagent at a molecular level to a surface. For example, a capture reagent may be immobilized to a surface of the substrate material using adsorption techniques including non-covalent interactions (e.g., electrostatic forces, van der Waals, and dehydration of hydrophobic interfaces) and covalent binding techniques where functional groups or linkers facilitate attaching the capture reagent to the surface. Immobilizing a capture reagent to a surface of a substrate material may be based upon the properties of the substrate surface, the medium carrying the capture reagent, and the properties of the capture reagent. In some cases, a substrate surface may be first modified to have functional groups bound to the surface. The functional groups may then bind to biomolecules or biological or chemical substances to immobilize them thereon.

The term “nucleic acid” generally refers to a set of nucleotides connected to each other via phosphodiester bond and refers to a naturally occurring nucleic acid to which a naturally occurring nucleotide existing in nature is connected, such as DNA comprising deoxyribonucleotides having any of adenine, guanine, cytosine, and thymine connected to each other and/or RNA comprising ribonucleotides having any of adenine, guanine, cytosine, and uracil connected to each other. In addition, non-naturally occurring nucleotides and non-naturally occurring nucleic acids are within the scope of the nucleic acid of the present invention. Examples include peptide nucleic acids (PNA), peptide nucleic acids with phosphate groups (PHONA), bridged nucleic acids/locked nucleic acids (BNA/LNA), and morpholino nucleic acids. Further examples include chemically-modified nucleic acids and nucleic acid analogues, such as methylphosphonate DNA/RNA, phosphorothioate DNA/RNA, phosphoramidate DNA/RNA, and 2′-O-methyl DNA/RNA. Nucleic acids include those that may be modified. For example, a phosphoric acid group, a sugar, and/or a base in a nucleic acid may be labeled as necessary. Any substances for nucleic acid labeling known in the art can be used for labeling. Examples thereof include but are not limited to radioactive isotopes (e.g., 32P, 3H, and 14C), DIG, biotin, fluorescent dyes (e.g., FITC, Texas, cy3, cy5, cy7, FAM, HEX, VIC, JOE, Rox, TET, Bodipy493, NBD, and TAMRA), and luminescent substances (e.g., acridinium ester).

The term “aptamer” as used herein refers to oligonucleic acids or peptide molecules that bind to a specific target molecule. The concept of using single-stranded nucleic acids (aptamers) as affinity molecules for protein binding was initially disclosed in 1990 (Ellington and Szostak 1990, 1992; Tuerk and Gold 1990), and is based on the ability of short sequences to fold, in the presence of a target, into unique, three-dimensional structures that bind the target with high affinity and specificity. Eugene W. M Ng et al., 2006, discloses that aptamers are oligonucleotide ligands that are selected for high-affinity binding to molecular targets.

The term “antibody” as used herein refers to a polypeptide of the immunoglobulin family that is capable of binding a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1 CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, and FR4. The three CDRs constitute about 15-20% of the variable domains. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. (Kuby, Immunology, 4th ed., Chapter 4. W.H. Freeman & Co., New York, 2000).

The term “antibody” includes, but is not limited to, monoclonal antibodies, human antibodies, humanized antibodies, chimeric antibodies, and anti-idiotypic (anti-Id) antibodies (including, e.g., anti-Id antibodies to antibodies of the invention). The antibodies can be of any isotype/class (e.g., IgG, IgE, IgM, IgD, IgA and IgY), or subclass (e.g., IgG1, IgG2, IgG3, IgG4, IgA1 and IgA2.

The term “polymer” means any substance or compound that is composed of two or more building blocks (‘mers’) that are repetitively linked to each other. For example, a “dimer” is a compound in which two building blocks have been joined together. Polymers include both condensation and addition polymers. Typical examples of condensation polymers include polyamide, polyester, protein, wool, silk, polyurethane, cellulose, and polysiloxane. Examples of addition polymers are polyethylene, polyisobutylene, polyacrylonitrile, poly(vinyl chloride), and polystyrene. Other examples include polymers having enhanced electrical or optical properties (e.g., a nonlinear optical property) such as electroconductive or photorefractive polymers. Polymers include both linear and branched polymers.

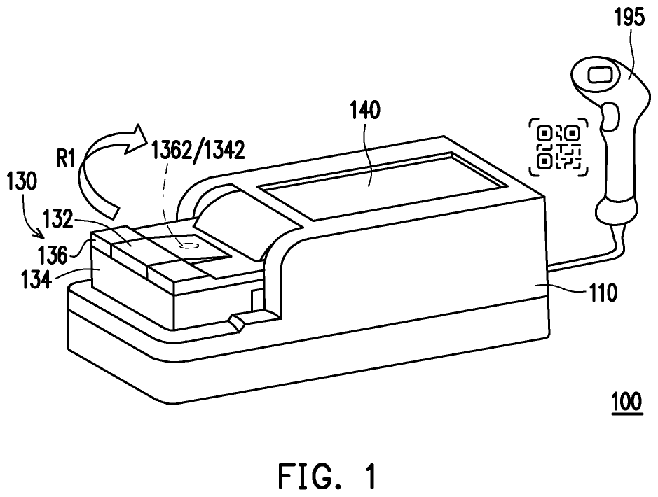

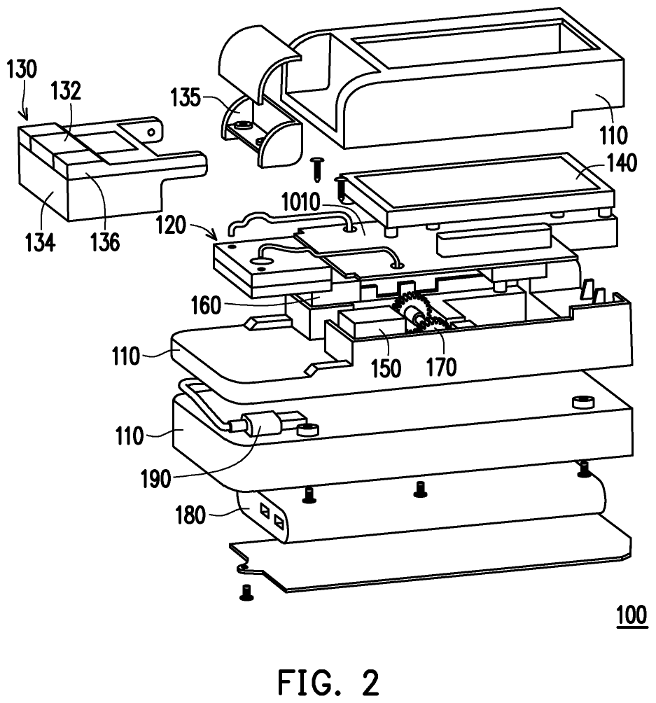

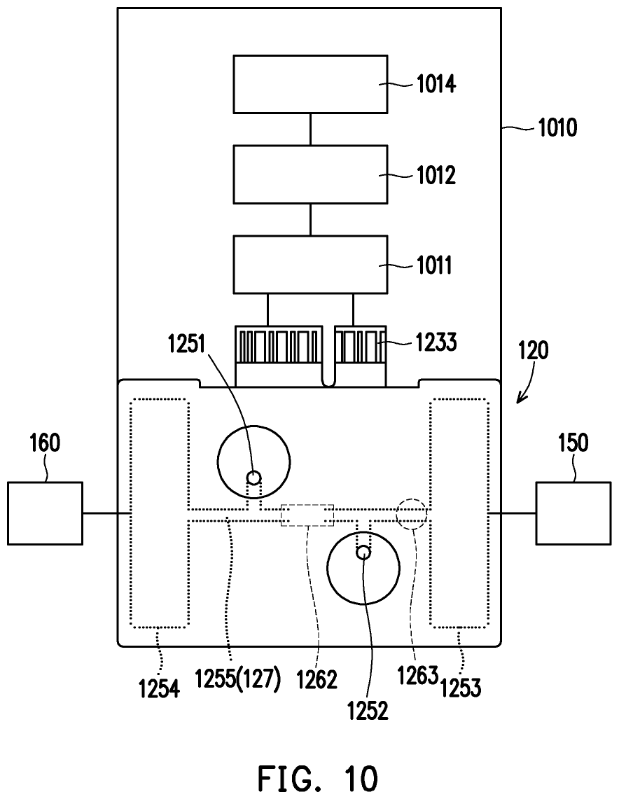

FIG. 1 illustrates a schematic view of a biosensor device according to some exemplary embodiments of the present disclosure. FIG. 2 illustrates an exploded view of a biosensor device according to some exemplary embodiments of the present disclosure. FIG. 1 and FIG. 2 illustrates an overview of various components that are integrated together to form a biosensor device 100. In some embodiments, the biosensor device 100 may include a housing 110, a fluidic cartridge module 120, and a processor, e.g., the processor 1012 shown in FIG. 10, mounted on a circuit board 1010. In some embodiments, the fluidic cartridge module 120 is disposed on the housing 110 and the processor is electrically coupled to the fluidic cartridge module 120 and disposed on the circuit board 1010. In some embodiments, the biosensor device 100 may further include a touch screen 140 disposed on the housing 110 and electrically coupled to the processor on the circuit board 1010. In some embodiments, the biosensor device 100 is configured to detects biomaterial on a biosensor chip of the fluidic cartridge module 120, and the touch screen allows a user to control the operation of the biosensor device 100, confirm an analysis result, or the like.

In accordance with some embodiments of the disclosure, the biosensor device 100 further includes a lid 130 pivotally connected to the fluidic cartridge module 120 through a rotating base 135. The lid 130 is configured to rotate relatively to the fluidic cartridge module 120 along a rotating direction R1. To be more specific, in one embodiment, the lid 130 may include an upper lid 132, and a lower lid 134. The lower lid 134 covers the fluidic cartridge module 120 and includes a buffer opening, and a sample 1342 opening respectively exposing a corresponding buffer inlet and a corresponding sample inlet of the fluidic cartridge module 120, such that a sample can flow into the fluidic cartridge module 120 through the sample opening 1342. In some embodiments, the lid 130 may further include a middle lid 136 having a sample opening 1362 corresponding to the sample opening 1342. The middle lid 136 may cover the buffer opening of the lower lid 134 to avoid user accidentally put the sample through the buffer opening. In some embodiments, the upper lid 132 is pivotally connected to the lower lid 134 to rotate relatively to the lower lid 134 along the rotating direction R1, such that the upper lid 132 can cover or exposing the sample opening 1362 underneath. In some scenarios, the lower lid 134 and the middle lid 136 may also be rotated along with the upper lid 132 to exposed the fluidic cartridge module 120 underneath.

In some embodiments, the biosensor device 100 may further include a power source 180 and a connector 190. The biosensor device 100 can be powered by the power source 180, such as a battery. In some embodiments, the power source 180 can be rechargeable, for example, using via inductive or wireless methods. In some embodiments, the patient can recharge the power source 180 when the appliance is not use through the connector 190 such as a USB connector, or the like. In other embodiments, the connector 190 may be connected to an external device such as a computer for building a connection (communication) between the biosensor device 100 and the external device.

In some embodiments, the biosensor device 100 may further include a scanner 195, while a 2D code such as barcode, QR code, or the like, may be printed, or otherwise applied, on a sample container containing the sample. Accordingly, the scanner 195 can scan the 2D code on the sample container to obtain various information about the sample contained therein. For example, the information may include patient's name, sample collecting date, total volume, etc. The 2D code encoding the necessary or desired information on the sample container may be scanned prior to the sample examination.

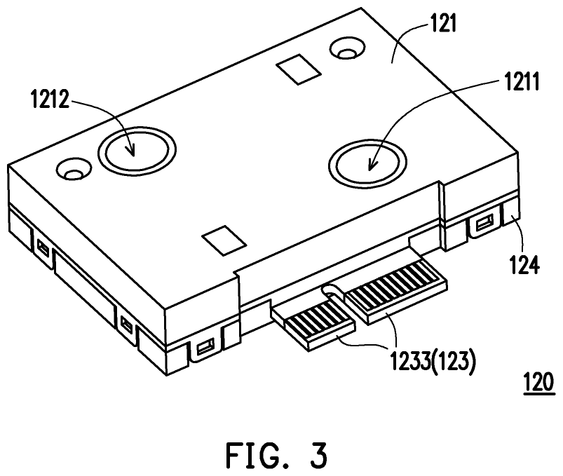

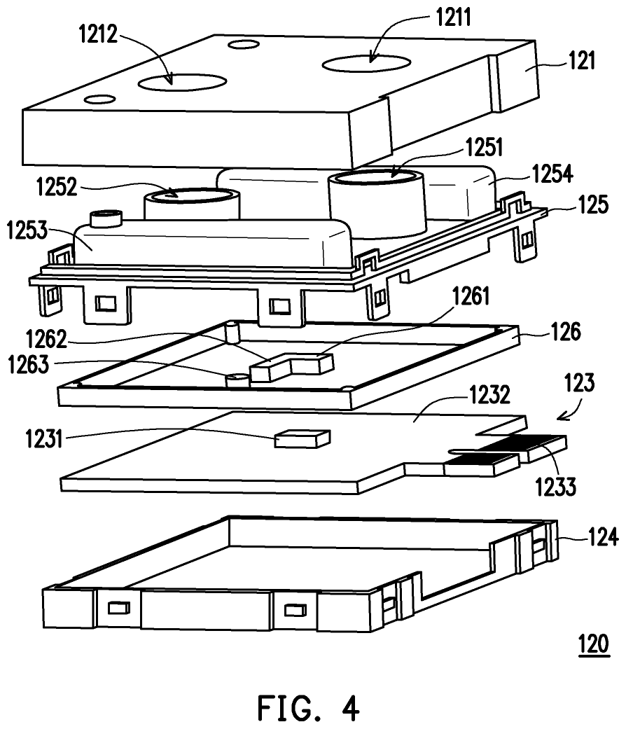

FIG. 3 illustrates a schematic view of a fluidic cartridge module according to some exemplary embodiments of the present disclosure. FIG. 4 illustrates an exploded view of the fluidic cartridge module in FIG. 3. Referring to FIG. 3 and FIG. 4, in some embodiments, the fluidic cartridge module 120 includes a casing 121, 122, a biosensor package 123, and a fluidic channel (e.g., the fluidic channel 127 shown in FIG. 10) that may be jointly formed (defined) by a first channel frame 126 and a second channel frame 125. The casing may be formed from any plastic material, such as polymethyl methacrylate (PMMA), using injection molding, casting, or 3-D printing techniques, to name a few examples. In some embodiments, the casing 121, 122 may be formed from more than one segment that connects together either mechanically or through the use of an adhesive. In one embodiment, the fluidic channel 127 and chambers 1253, 1254 are molded within one or more components (e.g., first channel frame 126 and second channel frame 125) of the casing. In another embodiment, the various fluidic channels and chambers are formed from a different molded polymer material, such as polydimethylsiloxane (PDMS). In an embodiment, the substrate 1232 having the biosensor chip 1231 is disposed within the casing 121, 122. In one example, at least a part of the portion of the substrate 1232 is enclosed within the casing 121, 122 while the edge connectors 1233 are exposed outside of the casing 121, 122.

In some embodiments, the casing includes a buffer inlet 1211, and a sample inlet 1212. In one embodiment, the casing may further include an upper casing 121 and a lower casing 122. The sample inlet 1212 and the buffer inlet 1211 are disposed on the upper casing 121 for receiving a sample fluid and a buffer fluid (solution) respectively. In some embodiments, the biosensor package 123 disposed in the casing 121, 122 and may include a substrate 1232 and a biosensor chip 1231 mounted on the substrate 1232. In some embodiments, the substrate 1232 includes an edge connector 1233 configured to be coupled to a circuit board, e.g., the circuit board 1010 shown in FIG. 2, such that the biosensor chip 1231 is electrically connected to the processor on the circuit board 1010 through the edge connector 1233.

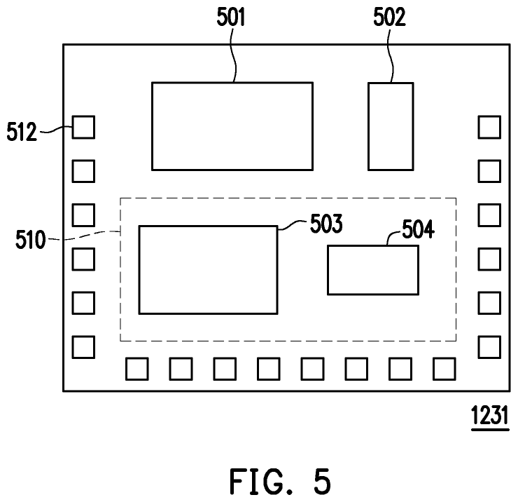

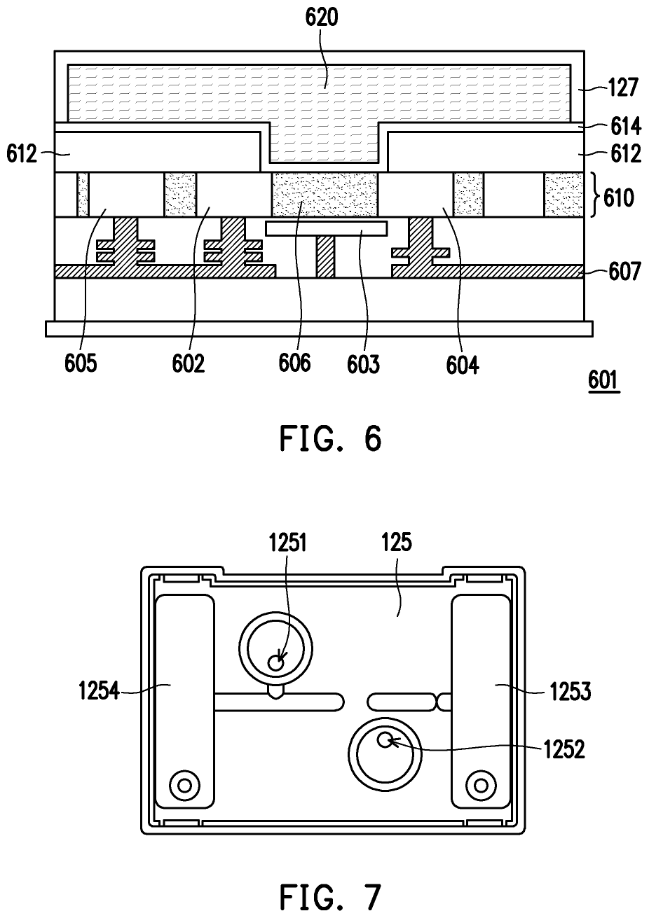

FIG. 5 illustrates a schematic floor plan diagram of a biosensor chip according to some exemplary embodiments of the present disclosure. FIG. 6 illustrates a schematic cross sectional view of a sensor of a biosensor chip according to some exemplary embodiments of the present disclosure. Referring firstly to FIG. 5, in some embodiments, an exemplary floor plan for a biosensor chip 1231 is shown. In the present embodiment, the biosensor chip 1231 may include a sensor array 501, a reference electrode 502, an analog circuitry 510, and an I/O pads 512. In some embodiments, the sensor array 501 may represent an array of dual gate back-side sensing FET sensors such as those illustrated hereinafter in FIG. 5. The array may be arranged as a row-column matrix of pixels. The various FET sensors in sensor array 501 may be functionalized with the same or different capture reagents to perform biosensing for various analytes.

In some embodiments, the reference electrode 502 may be patterned on the same biosensor chip 1231 that includes the sensor array 501. In one embodiment, the reference electrode 502 may be roughly aligned with sensor array 501 along an X or Y direction, such that the fluidic channel may be placed over both the sensor array 501 and the reference electrode 502. In another embodiment, the reference electrode 502 may be provided elsewhere off of biosensor chip 1231. The reference electrode 502 may include any material having a relatively stable potential. Example reference electrode materials include platinum or Ag/AgCl.

In accordance with some embodiments of the disclosure, the analog circuitry 510 may include circuitry related to the operation of the sensor array 501. As such, the analog circuitry 510 may be configured to provide signals to, and measure signals from, sensor array 501, while interfacing with various I/O pads 512. In one embodiment, the analog circuitry 510 may include a sensor array circuitry 503, and a serial peripheral interface (SPI) 504. SPI 503 may be a serial interface circuit to facilitate data transmission between sensor array circuitry 503 and an analyzer unit. The general operation of a SPI would be well understood to a person skilled in the relevant art. The sensor array circuitry 503 may include any number of reference voltage generators, operational amplifiers, low pass filters, ADCs, and DACs to provide signals to, and receive signals from, the sensor array 501.

In one embodiment, when measuring signals received from a given FET sensor or a set of FET sensors in sensor array 501, the sensor array circuitry 503 may receive the measured signals and pass them through a trans-impedance amplifier, i.e., a current-to-voltage converter, followed by one or more additional amplification stages, low pass filters, and ultimately an ADC, before the resulting signal is output to an I/O pad 512. Noise may also be reduced from the measured signal by subtracting a background AC signal from the measured signal before the measured signal is amplified. In various embodiments a plurality of I/O pads 512 may be patterned along the periphery of the biosensor chip 1231. In one embodiment, wire bonding techniques may be used to couple various I/O pads 512 to another substrate or package bonded to the biosensor chip 1231.

Referring now to FIG. 6, a cross section of an exemplary sensor 601 of the sensor array 501 is provided. In the present embodiment, the sensor 601 may be a FET (field-effect transistor) sensor 601. The FET sensors 601 makes up the transducer component of the fluidic cartridge module 120. The FET sensors 601 may be arranged in an array and individually addressed to detect binding events at the surface of the FET sensor sensing layer. In one embodiment, the FET sensors 601 includes dual gate back-side FET sensors. In alternative embodiments other types of FET sensor-based sensors may be used.

In some embodiments, the dual gate back-side sensing FET sensor 500 includes gate 603, source region 602, drain region 604, and channel region 606, where the source region 602 and the drain region 604 are formed within substrate 610. The gate 603, the source region 602, the drain region 604, and the channel region 606 form a FET. It should be noted that the various components in the figures are not intended to be drawn to scale and are exaggerated for visual convenience, as would be understood by a person skilled in the relevant art. In an exemplary embodiment, dual gate back-side sensing FET sensor 601 is coupled to various layers of metal interconnects 607 that make electrical connection with the various doped regions and other devices formed within substrate 610. Metal interconnects 607 may be manufactured using fabrication processes well known to a person skilled in the relevant art.

In some embodiments, the dual gate back-side FET sensor 601 may include a body region 605 separate from the source region 602 and the drain region 604. The body region 605 may be used to bias the carrier concentration in the channel region 606 between the source region 602 and the drain region 604. As such, a negative voltage bias may be applied to the body region 605 to improve the sensitivity of dual gate back-side FET sensor 601. In one embodiment, the body region 605 is electrically connected with the source region 602. In another embodiment, the body region 605 is electrically grounded.

The dual gate back-side FET sensor 601 may be coupled to additional circuitry fabricated within substrate 610. The circuitry may include any number of MOSFET devices, resistors, capacitors, or inductors to form circuitry to aid in the operation of dual gate back-side sensing FET sensor 601. The circuitry may include any amplifiers, analog to digital converters (ADCs), digital to analog converters (DACs), voltage generators, logic circuitry and DRAM memory, to name a few examples. All or some of the components of additional circuitry may be integrated in the same substrate 610 as dual gate back-side FET sensor 601. It should be understood that many FET sensors, each substantially similar to dual gate back-side FET sensor 601, may be integrated on substrate 610 and coupled to additional circuitry.

Still referring to the illustrative example of FIG. 6, the dual gate back-side sensing FET sensor 601 includes an interface layer 614 deposited over isolation layer 612 and within the opening over channel region 606. In one embodiment, the interface layer 614 may be a high-K dielectric material, such as hafnium silicate, hafnium oxide, zirconium oxide, aluminum oxide, tantalum pentoxide, hafnium dioxide-alumina (HfO₂—Al₂O₃) alloy, or any combinations thereof. The interface layer 614 may act as a support for the attachment of capture reagents as will be discussed in more detail later in the section directed to biological sensing.

An example operation of dual gate back-side FET sensor 601 may act as a pH sensor will now be described. Briefly, a solution 620 having a given pH is provided over the reaction site of dual gate back-side sensing FET sensor 601 through the fluidic channel 127. The pH of the solution is generally related to the concentration of hydrogen ions [H+] in the solution. The accumulation of the ions near the surface of the interface layer 614 above the channel region 606 will affect the formation of the inversion layer within channel region 606 that forms the conductive pathway between the source region 602 and the drain region 206. This can be measured by the change in the conductivity of the FET sensor.

With now reference to FIG. 4 and FIG. 5, in some embodiments, the biosensor chip 1231 is mounted on the substrate 1232. The substrate 1232 may be a printed circuit board (PCB), or the like. A flip-chip bonding technique may be performed to bond the biosensor chip 1231 onto the surface of substrate 1232. The I/O pads from the biosensor chip 1231 is bonded to conductive traces present on the substrate 1232. The conductive traces on substrate 902 may terminate in the edge connectors 1233. In some embodiments, one or more edge connectors 1233 may provide electrical connection to the biosensor chip 1231. In some embodiments, one or more other edge connectors 1233 may provide electrical connection to the reference electrode 502 patterned on the substrate 1232. Each of the one or more edge connectors 1233 may be patterned using a metal such as, but not limited, copper, gold, or aluminum. In one embodiment, the reference electrode 502 may be formed on the biosensor chip 1231. In an alternative embodiment, the reference electrode 502 may be formed on the substrate 1232. In some embodiments, the sample inlet 1212 disposed on the upper casing 121, and the sample inlet 1252 disposed on the second channel frame 125 are located over the biosensor chip 1231, and in fluid communication with at least the sensor array 501 of biosensor chip 1231. The buffer inlet 1211 disposed on the upper casing 121, and the sample inlet 1251 disposed on the second channel frame 125 are located over the biosensor chip 1231, and in fluid communication with at least the reference electrode 502 of biosensor chip 1231.

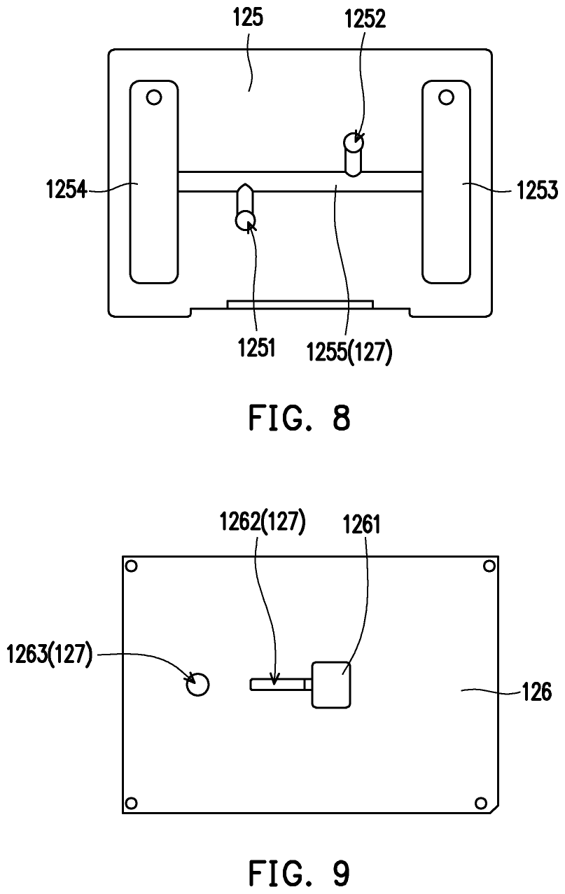

With now reference to FIG. 7 to FIG. 10, in some embodiments, the fluidic channel 127 disposed over the biosensor package 123 is jointly defined by the first channel frame 126 and the second channel frame 125. For example, the first channel frame 126 includes a first opening 1262 aligned with (exposing) the sensor array of the biosensor chip 123 and the second opening 1262 aligned with (exposing) the reference electrode of the biosensor chip 123. The second channel frame 125 includes a channel groove 1255 connected to the sample inlet 1252 and the buffer inlet 1251 and passing through the first opening 1262 and the second opening 1263. The fluidic channel 127 is configured to control fluid, e.g., sample fluid, buffer fluid, or the like, flow both towards and away from a sensing location, e.g., sensing array, where the presence of a target analyte can be detected. In some embodiments, the first channel frame 126 may further include a concave 1261 disposed on a lower surface of the first channel frame 126 for accommodating the biosensor chip 1231. In the illustrated embodiment, the concave 1261 may not penetrate (extend through) the first channel frame 126 while the first opening 1262 and the second opening 1261 penetrate (extend through) the first channel frame 126 for fluid communication.

Referring to FIG. 4 and FIG. 10, in some embodiments, the schematic illustrates a top-down view of the fluidic cartridge module 120 coupled to a circuit board 1010, and it should be noted that not all elements shown are on the same horizontal plane. Also, the specific dimensions and scale of the various fluidic channels are purposefully not drawn to scale for improved visualization. In some embodiments, the buffer inlet 1251 and the sample inlet 1252 provide area for the buffer fluid and the sample fluid to flow into the fluidic channel 127 from outside of the fluidic cartridge module 120. In some embodiments, the fluidic cartridge module 120 may further include at least one discharged chamber. In the illustrated embodiment, two discharged chambers 1253, 1254 are illustrated herein, but less or more of the discharged chambers may be incorporated. The discharged chambers 1253, 1254 collect fluids flowing through the fluidic channel 127. the fluidic channel 127 is in fluid communication with the discharged chambers 1253, 1254 to expel fluid from the fluidic channel 127 to outside of the fluidic cartridge module 120. In some embodiments, the channel groove 1255 of the second channel frame 125 that forms a part of the fluidic channel 127 may be aligned over the biosensor chip 1231. In one embodiment, the first opening 1262 of the first channel frame 126 over the sensor array (e.g., the sensor array 501 shown in FIG. 5) is in fluid communication with the fluidic channel 127. The reference electrode (e.g., the reference electrode 502 shown in FIG. 5) may be aligned with the second opening 1261 in fluid communication with the fluidic channel 127, according to an embodiment.

Accordingly, the fluid flowing from the buffer inlet 1251 and the sample inlet 1252 will flow eventually through the fluidic channel 127. In some embodiments, the fluidic channel 127 eventually flows into the discharged chambers 1253, 1254 that collects all fluids flowing through the fluidic cartridge module 120. The discharged chambers 1253, 1254 may include a vent to the atmosphere to avoid backpressure building up within the fluidic cartridge module 120.

Referring to FIG. 10, in some embodiments, the fluidic cartridge module 120 is coupled to the circuit board 1010 through the edge connectors 1233. The electrical connections made to edge connectors 1233 may be routed to the sensing electronics 1011 of the circuit board 1010. The sensing electronics 1011 may include any number of discrete circuits, integrated circuits, and discrete analog circuit components that are designed to both provide and receive numerous different electrical signals between sensing electronics 1011 and edge connectors 1233. For example, sensing electronics 1011 may be configured to provide power, ground, and clock signals to edge connectors 1233, which may be subsequently used to power and operate the sensor array and other electronics on the biosensor chip 1231. The sensing electronics 1011 may also provide various voltage bias levels for activating the gates of particular FET sensors within the sensor array. The sensing electronics 1011 may receive signals that represent drain currents measured from particular FET sensors, and signals that represent outputs from temperature sensors on the biosensor chip 1231. The sensing electronics 1011 may store this received data in a memory, or may use the received data to alter the voltage bias levels, or to change an amount of heat generated by heaters on the biosensor chip 1231. Generally, the sensing electronics 1011 control all signaling related to the biosensing performed by the sensor array of fluidic cartridge module 120.

In some embodiments, the processor 1012 is configured to determine a concentration level of a given analyte from the sample fluid in the fluidic cartridge module 120 based on signals received from the sensor array. The processor 1012 may be any type of central processing unit (CPU) or microcontroller and may be programmable by a user to perform certain functions. Processor 1012 may be configured to analyze signals received from sensing electronics 1011 to determine a concentration level of a given analyte from the sample in the fluidic cartridge module 120. Data related to the determined concentration levels may be stored in a memory of the circuit board.

In some embodiments, the biosensor device 100 may further include a communication module 1014 that is designed to communicate data to an external processing device. The processor 1012 may be electrically coupled with the communication module 1014 to control data transfer. The communication may be wired or wireless. Examples of wired communication include data transfer via a network cable or a universal serial bus (USB) cable. Wireless communication may include radio RF transmission, Bluetooth, WiFi, 3G, or 4G. The communication module 1014 may also be designed to receive data from the external processing device. For example, a program for how to operate the various components of the biosensor device 100 may be transmitted to communication module 1014 and executed by the processor 1012. The communication module 1014 may include any number of well-known hardware elements to facilitate analog and/or digital data transmission and reception.

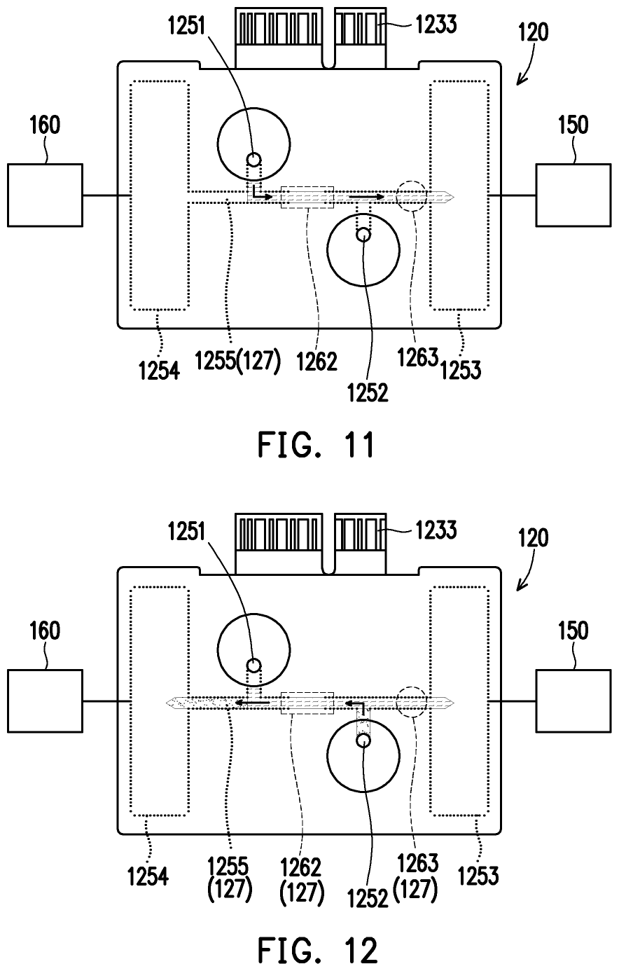

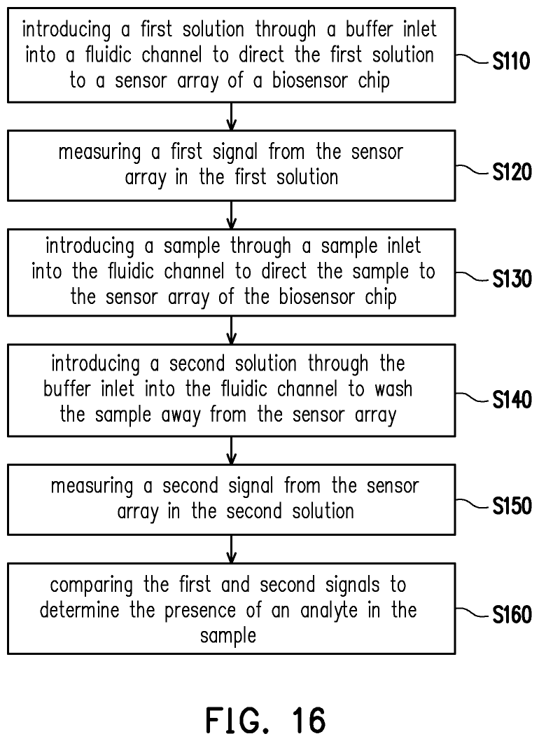

FIG. 11 to FIG. 13 illustrate schematic operating scenarios of a fluidic cartridge module according to some exemplary embodiments of the present disclosure. FIG. 16 illustrates a flow diagram of an exemplary method of detecting an analyte in a sample. With the configuration described above, a method of detecting an analyte in a sample using the biosensor device 100 is developed, and the method may include the following steps. Referring firstly to FIG. 11 and FIG. 16, at step S110, a first solution may be introduced through a buffer inlet 1251 into the fluidic channel 127 to direct the first solution to the sensor array (e.g., the sensor array 501 shown in FIG. 5) of the biosensor chip (e.g., the biosensor chip 1231 shown in FIG. 5). The first solution may enter the fluidic channel 127 via the buffer inlet 1251 coupled to the fluidic channel 127. The first solution may include a buffer solution to provide a stable pH environment, such as phosphate buffered saline (PBS), MES, or the like. In some embodiments, the first solution may be moved along the fluidic channel 127 using pressure driven flow. Accordingly, the biosensor device 100 may further include a first pump 150 in fluid communication with the fluidic channel 127 for driving a buffer fluid (e.g., first solution) from the buffer inlet 1251 to flow passing the first opening 1262 and the second opening 1263.

Then, at block S120, a first signal is measured from the sensor array in the first solution. In some embodiment, the FET sensor of the sensor array are calibrated in the first solution, and the calibration is performed to measure a noise or background signal (i.e., first signal) of the various FET sensors. This measurement may be stored and later subtracted from the measured signal when detecting biomolecules to try and reduce the noise and achieve a clearer detection signal. The first solution must be present over the sensor array and the reference electrode patterned through the first opening 1262 and the second opening 1263 to perform the calibration.

Then, referring to FIG. 12 and FIG. 16, at block S130, a sample fluid is introduced through the sample inlet 1252 into the fluidic channel 127 to direct the sample fluid to the sensor array of the biosensor chip. In the illustrated embodiment, the sample fluid may be any liquid sample, including blood, plasma, serum, urine, saliva or sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid and combinations thereof. In other embodiments, the sample fluid may be a semi-solid sample that dissociates within solution. In some embodiments, after the sample fluid has been input via the sample inlet 1252, the sample inlet 1252 may be sealed by the lid, a cap, or other similar structure. In some embodiments, the sample fluid may be moved along the fluidic channel 127 using pressure driven flow. Accordingly, the biosensor device 100 may further include a second pump 160 in fluid communication with the fluidic channel 127 for driving the sample fluid from the sample inlet 1252 to flow passing the first opening 1262 and the second opening 1263. In the illustrated embodiment, the first pump 150 and the second pump 160 are in fluid communication with the discharged chambers 1253, 1254 respectively.

In one embodiment, then, a buffer fluid may flow through the fluidic channel 127. The buffer fluid may be the same solution as the first solution. The buffer fluid may cross paths with the sample fluid flowing into the fluidic channel 127 at block S130, and mix with the sample fluid. The mixture of the sample fluid and the buffer fluid may then flow to the sensor array. The buffer fluid may be moved along and between the fluidic channel 127 a driven of the second pump.

In some embodiments, the biomolecules present within the sample fluid are incubated over the sensor array for a given period of time. Incubation may last for any given amount of time, for example, between 30 seconds and 10 minutes. During incubation, the sample fluid mixed with the buffer fluid may not be flowing, or may be flowing at a very slow flow rate. The flow rate may be designed such that fresh solution is presented over the sensor array over time, but the flow is not too strong to cause damage to the capture reagents or to not allow for the binding reactions to occur.

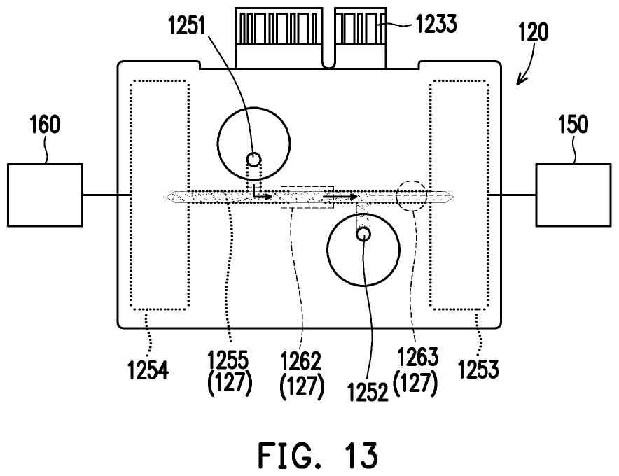

Then, referring to FIG. 13 and FIG. 16, at block S140, after the incubation time has expired, a second solution is introduced through the buffer inlet 1251 into the fluidic channel 127 to wash the sample away from the sensor array. In some embodiments, the second solution flows through the fluidic channel 127 to push substantially all of the sample mixed with the buffer solution into the discharged chamber 1253 and/or discharged chamber 1254. The second solution may be injected through the fluidic channel 127 for a given period of time to ensure that the sample has been cleared from the fluidic channel 127. The second solution used in block S140 should ideally be the same solution as the first solution. In another embodiment, the second solution is different from the first solution. The second solution may be a buffer solution.

Then, at block S150, a second signal is measure from the sensor array in the second solution. For example, the output from the sensor array (i.e., second signal) is measured to determine if any binding reactions occurred. The sensor output may be a drain current measured from one or more of the dual gate back-side sensing FET sensors in the sensor array. Then, at block S160, the first and second signals are compared to determine the presence of an analyte in the sample by the processor. For example, the measured drain current may be compared to a drain current measured during calibration of the same sensor in block S120. If the threshold voltage (e.g., roughly corresponding to the voltage needed to turn on the FET and cause the drain current to flow) has changed from when the sensor was calibrated, then it may be determined that a binding reaction has occurred and that a target analyte was present in the sample. In another example, the measured output from the sensor array is the threshold voltage itself, which may be compared to a threshold voltage measured during calibration of the same sensor in block S120.

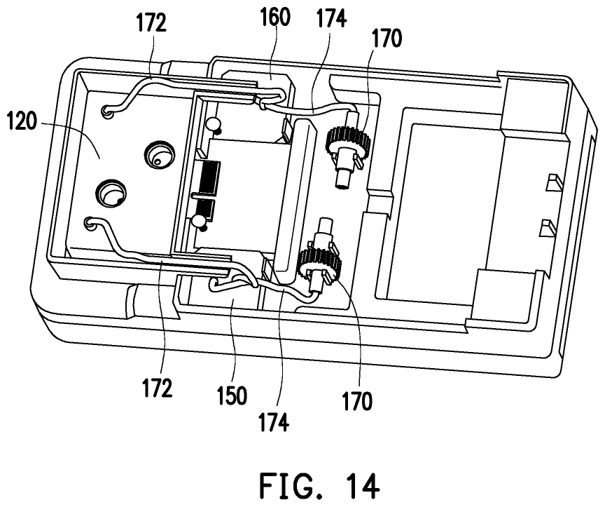

FIG. 14 illustrates a schematic view of a part of a biosensor device according to some exemplary embodiments of the present disclosure. With now reference to FIG. 13 and FIG. 14, in some embodiments, the biosensor device 100 may further include at least one filter 170 (two filters 170 are illustrated, but not limited thereto). In one embodiment, the filter 170 is disposed in the housing 110 and in fluid communication with the discharged chamber 1253, 1254 for filtering the fluids from the discharged chamber 1253, 1254. In the illustrated embodiment, the discharged chamber 1253, 1254 is coupled to the first pump 150 and the second pump 160 respectively through the pipes 172, so the fluid in the fluidic cartridge module 120 can be driven by the first pump 150 and the second pump 160. The discharged chamber 1253, 1254 may also be coupled to the filter 170 respectively through the pipes 174, so the fluid from the discharged chamber 1253, 1254 can be filtered before being discharged from the biosensor device 100 to avoid infection and/or contamination.

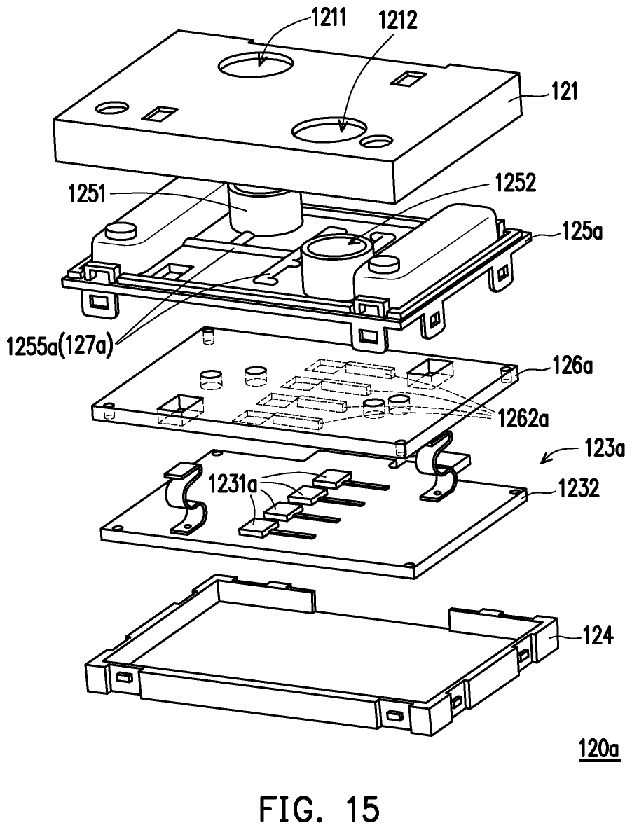

FIG. 15 illustrates an exploded view of the fluidic cartridge module according to some exemplary embodiments of the present disclosure. With now reference to FIG. 15, in some embodiments, the biosensor package 123a may include a plurality of biosensor chips 1231a mounted on the substrate of the biosensor package 123a. Accordingly, the fluidic channel 127a may include a plurality of first openings 1262a, which are aligned with a plurality of sensor arrays of the biosensor chips 1231a respectively. For example, each of the biosensor chips 1231a may be configured to receive different samples from different patients or the same sample for various testing results. For example, in the illustrated embodiment, the biosensor package 123a includes 4 biosensor chips 1231a, and each of the biosensor chips 1231a may serve for different purposes, such as positive detection of Covid-19, positive detection of Covid-19, no antigen detection, and SARS detection, etc. purified recombinant proteins, such as covid-19, SARS, H1N1, etc., as well as positive control proteins and negative control, were tested. As an example, anti-his-tag antibodies were used as positive control for his-tagged recombinant proteins. Examples of negative controls include BSA, his tagged only, MBP only, buffer only, etc. Initially, the conditions were optimized using obtained antibodies, such as monoclonal and polyclonal antibodies against E6, E7, L1 proteins. Varied concentrations of the purified recombinant proteins spotted on the protein chips were used to maximize binding. A secondary antibody coupled to, for example, Cy3 or Cy5, was added to the surface of the protein chips to increase sensitivity. Assay specificity and sensitivity were obtained. Positive controls and negative controls from cell culture samples or clinical samples were also tested.

With such device and method, the biosensor device 100 adopts bio-sensitive field-effect transistors (Bio-FETs) to combine surface chemistry to immobilize the antibody on chip surface for diseases detection by one-button automated electrical bio-detector, which enables the more efficient design of clinical trials, so that the test result can be generated within a few minutes (under 10 minutes), and does not need to be operated by professionals. In addition, the biosensor device 100 is proved to be capable of detecting 0.01, 0.1, 1 and 10 fg/mL concentrations of SARS-CoV-2 nucleoprotein antigen within 10 minutes, and the experimental data showed the detection of limitation (LoD) was 0.1 fg/mL concentration of SARS-CoV-2 nucleoprotein antigen. Therefore, the biosensor device 100 is portable, label free, and one-button activated, which can provide a faster, lower cost, and more accurate method with low LoD for urgent situations and early diagnosis.

Based on the above discussions, it can be seen that the present disclosure offers various advantages. It is understood, however, that not all advantages are necessarily discussed herein, and other embodiments may offer different advantages, and that no particular advantage is required for all embodiments.

In accordance with some embodiments of the disclosure, a fluidic cartridge module includes a casing, a biosensor package, and a fluidic channel. The casing includes a sample inlet and a buffer inlet, a biosensor package disposed in the casing and comprising a sensor array and a reference electrode. The fluidic channel is disposed over the biosensor package and connected to the sample inlet and the buffer inlet, wherein the fluidic channel includes a first opening aligned with the sensor array and a second opening aligned with the reference electrode. In one embodiment, the fluidic cartridge module further includes a discharged chamber collecting fluids flowing through fluidic channel. In one embodiment, the biosensor package further includes a substrate and a biosensor chip mounted on the substrate, and the substrate includes an edge connector electrically connected to the biosensor chip and the processor. In one embodiment, the biosensor package includes a plurality of biosensor chips, and the fluidic channel comprises a plurality of first openings aligned with a plurality of sensor arrays of the plurality of biosensor chips respectively. In one embodiment, the fluidic cartridge module further includes a first channel frame disposed over the biosensor package and a second channel frame disposed over the first channel frame, wherein the first channel frame and the second channel frame jointly form the fluidic channel. In one embodiment, the first channel frame includes the first opening and the second opening, and the second channel frame includes a channel groove connected to the sample inlet and the buffer inlet and passing through the first opening and the second opening. In one embodiment, the biosensor package further includes a substrate and a biosensor chip mounted on the substrate, and the first channel frame further includes a concave disposed on a lower surface of the first channel frame for accommodating the biosensor chip.

In accordance with some embodiments of the disclosure, a biosensor device includes a housing, a fluidic cartridge module, and a processor. The fluidic cartridge module is disposed on the housing and includes a sample inlet, a buffer inlet, a biosensor package including a sensor array and a reference electrode and a fluidic channel disposed over the biosensor package and connected to the sample inlet and the buffer inlet, wherein the fluidic channel includes a first opening aligned with the sensor array and a second opening aligned with the reference electrode. The processor is disposed in the housing electrically coupled to the fluidic cartridge module, wherein the processor is configured to determine a concentration level of a given analyte from the sample fluid in the fluidic cartridge module based on signals received from the sensor array. In one embodiment, the biosensor device further includes a first pump in fluid communication with the fluidic channel for driving a buffer fluid from the buffer inlet to flow passing the first opening and the second opening; and a second pump in fluid communication with the fluidic channel for driving a sample fluid from the sample inlet to flow passing the first opening and the second opening. In one embodiment, the fluidic cartridge module further includes a discharged chamber collecting fluids flowing through fluidic channel. In one embodiment, the biosensor device further includes a filter disposed in the housing and in fluid communication with the discharged chamber for filtering the fluids from the discharged chamber. In one embodiment, the biosensor device further includes a lid pivotally connected to the housing and configured to rotate relatively to the fluidic cartridge module. In one embodiment, the biosensor device further includes a touch screen disposed on the housing and electrically coupled to the processor. In one embodiment, the biosensor package further includes a substrate and a biosensor chip mounted on the substrate, and the substrate includes an edge connector electrically connected to the biosensor chip and the processor. In one embodiment, the biosensor package includes a plurality of biosensor chips, and the fluidic channel includes a plurality of first openings aligned with a plurality of sensor arrays of the plurality of biosensor chips respectively.

In accordance with some embodiments of the disclosure, a method of detecting an analyte in a sample includes the following steps. A first solution is introduced through a buffer inlet into a fluidic channel to direct the first solution to a sensor array of a biosensor chip. A first signal is measured from the sensor array in the first solution. A sample is introduced through a sample inlet into the fluidic channel to direct the sample to the sensor array of the biosensor chip. A second solution is introduced through the buffer inlet into the fluidic channel to wash the sample away from the sensor array. A second signal is measured from the sensor array in the second solution. The first and second signals are compared to determine the presence of an analyte in the sample. In one embodiment, the method further includes incubating the sample over the sensor array for a given period of time before introducing the second solution to wash the sample away. In one embodiment, the first solution and the second solution are moved along the fluidic channel by a driven of a first pump. In one embodiment, the sample is moved along the fluidic channel by a driven of a second pump. In one embodiment, comparing the first and second signals are performed by a processor coupled to a fluidic cartridge module that comprises the fluidic channels and the sensor array.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A fluidic cartridge module, comprising:
a casing comprising a sample inlet and a buffer inlet;
a biosensor package disposed in the casing and comprising a sensor array and a reference electrode; and
a fluidic channel disposed over the biosensor package and connected to the sample inlet and the buffer inlet, wherein the fluidic channel comprises a first opening aligned with the sensor array and a second opening aligned with the reference electrode.

2. The fluidic cartridge module as claimed in claim 1, wherein the fluidic cartridge module further comprises a discharged chamber collecting fluids flowing through fluidic channel.

3. The fluidic cartridge module as claimed in claim 1, wherein the biosensor package further comprises a substrate and a biosensor chip mounted on the substrate, and the substrate comprises an edge connector electrically connected to the biosensor chip and the processor.

4. The fluidic cartridge module as claimed in claim 1, wherein the biosensor package comprises a plurality of biosensor chips, and the fluidic channel comprises a plurality of first openings aligned with a plurality of sensor arrays of the plurality of biosensor chips respectively.

5. The fluidic cartridge module as claimed in claim 1, further comprising a first channel frame disposed over the biosensor package and a second channel frame disposed over the first channel frame, wherein the first channel frame and the second channel frame jointly form the fluidic channel.

6. The fluidic cartridge module as claimed in claim 1, wherein the first channel frame comprises the first opening and the second opening, and the second channel frame comprises a channel groove connected to the sample inlet and the buffer inlet and passing through the first opening and the second opening.

7. The fluidic cartridge module as claimed in claim 1, wherein the biosensor package further comprises a substrate and a biosensor chip mounted on the substrate, and the first channel frame further comprising a concave disposed on a lower surface of the first channel frame for accommodating the biosensor chip.

8. A biosensor device, comprising:
a housing;
a fluidic cartridge module disposed on the housing and comprising a sample inlet, a buffer inlet, a biosensor package comprising a sensor array and a reference electrode and a fluidic channel disposed over the biosensor package and connected to the sample inlet and the buffer inlet, wherein the fluidic channel comprises a first opening aligned with the sensor array and a second opening aligned with the reference electrode; and
a processor disposed in the housing electrically coupled to the fluidic cartridge module, wherein the processor is configured to determine a concentration level of a given analyte from the sample fluid in the fluidic cartridge module based on signals received from the sensor array.

9. The biosensor device as claimed in claim 8, further comprising:
a first pump in fluid communication with the fluidic channel for driving a buffer fluid from the buffer inlet to flow passing the first opening and the second opening; and
a second pump in fluid communication with the fluidic channel for driving a sample fluid from the sample inlet to flow passing the first opening and the second opening.

10. The biosensor device as claimed in claim 8, wherein the fluidic cartridge module further comprises a discharged chamber collecting fluids flowing through fluidic channel.

11. The biosensor device as claimed in claim 10, further comprising a filter disposed in the housing and in fluid communication with the discharged chamber for filtering the fluids from the discharged chamber.

12. The biosensor device as claimed in claim 8, further comprising a lid pivotally connected to the housing and configured to rotate relatively to the fluidic cartridge module.

13. The biosensor device as claimed in claim 8, further comprising a touch screen disposed on the housing and electrically coupled to the processor.

14. The biosensor device as claimed in claim 8, wherein the biosensor package further comprises a substrate and a biosensor chip mounted on the substrate, and the substrate comprises an edge connector electrically connected to the biosensor chip and the processor.

15. The biosensor device as claimed in claim 8, wherein the biosensor package comprises a plurality of biosensor chips, and the fluidic channel comprises a plurality of first openings aligned with a plurality of sensor arrays of the plurality of biosensor chips respectively.

16. A method of detecting an analyte in a sample, comprising:
introducing a first solution through a buffer inlet into a fluidic channel to direct the first solution to a sensor array of a biosensor chip;
measuring a first signal from the sensor array in the first solution;
introducing a sample through a sample inlet into the fluidic channel to direct the sample to the sensor array of the biosensor chip;
introducing a second solution through the buffer inlet into the fluidic channel to wash the sample away from the sensor array;
measuring a second signal from the sensor array in the second solution; and
comparing the first and second signals to determine the presence of an analyte in the sample.

17. The method of claim 16, further comprising incubating the sample over the sensor array for a given period of time before introducing the second solution to wash the sample away.

18. The method of claim 16, wherein the first solution and the second solution are moved along the fluidic channel by a driven of a first pump.

19. The method of claim 18, wherein the sample is moved along the fluidic channel by a driven of a second pump.

20. The method of claim 16, wherein comparing the first and second signals are performed by a processor coupled to a fluidic cartridge module that comprises the fluidic channels and the sensor array.
